(19) United States
(12) Patent Application Publication
(10) Pub. No.: US 2016/0214918 A1
(43) Pub. Date: Jul. 28, 2016

(54) COMPOSITION FOR TREATING METABOLIC SYNDROME AND A PREPARATION METHOD THEREOF

(71) Applicant: Kaohsiung Medical University, Kaohsiung (TW)

(72) Inventors: Fang-Rong Chang, Kaohsiung (TW); Yang-Chang Wu, Kaohsiung (TW); Tusty-Jiuan Hsieh, Kaohsiung (TW); Chi-Ting Hsieh, Kaohsiung (TW); Ferenc Fülöp, Kaohsiung (TW); Sándor Balázs Ötvös, Kaohsiung (TW)

(73) Assignee: Kaohsiung Medical University, Kaohsiung (TW)

(21) Appl. No.: 15/003,705

(22) Filed: Jan. 21, 2016

Related U.S. Application Data
(60) Provisional application No. 62/106,480, filed on Jan. 22, 2015.

(51) Int. Cl. C07C 49/84; C07C 49/794

(52) U.S. Cl. CPC C07C 49/84; C07C 49/794

(57) ABSTRACT

A compound for treating a metabolic syndrome, comprising a structure of a 6Cs unit-3Cs unit-6Cs unit (C6-C3-C6) as shown in formula I, wherein: when R₁ and R₂ are both hydrogens, C3 has only single bonds; when R₁ is an oxygen and R₂ is one selected from a group consisting of an alkoxy, a benzyloxy and a halogen, C3 has only single bonds; when R₁ is a deuterium and R₂ is one selected from a group consisting of a hydrogen, an alkoxy, a benzyloxy and a halogen, C3 has one of a single bond and a double bond.

### CROSS-REFERENCE TO RELATED APPLICATION AND CLAIM OF PRIORITY

This application claims the benefit of U.S. Provisional Patent Application No. 62/106,480, filed on Jan. 22, 2015, at the United States Patent and Trademark Office, the disclosures of which are incorporated herein in their entirety by reference.

### FIELD OF THE INVENTION

The present invention is related to a composition for treating one of metabolic syndromes and a preparation method thereof, and more particularly to a compound of a series of chalcones for treating diabetes mellitus.

### BACKGROUND OF THE INVENTION

According to forecasts form the World Health Organization (WHO), the number of patients with diabetes mellitus will reach 3.60 billion by 2030. Compared to the statistical data from 2000, it is estimated that the increase in the number of patients with diabetes mellitus in the United States will be rise 102% compared to the corresponding data for 2030.

Similarly, the increase in the number of the patients with diabetes mellitus is estimated at 43% in Europe and 130% in the Asia-Pacific region. The global number of patients with diabetes mellitus is rapidly increasing. Drug sales for diabetes mellitus are expected to reach 430˜480 billion dollars in the market for 2015 according to estimates.

The top three shares in the global drugs for diabetes mellitus in 2010 determined by the Development Center for Biotechnology are insulin and its analogues (52.8%), Glitazone (TZD like drugs, 17.2%) and Dipeptidyl peptidase 4 inhibitors, DPP 4 (10.4%).

However, drugs directed to peripheral tissues, such as adipose tissues and muscles, are only found in TZD types of drugs. However sales of this drug have been suspended because of side effects found in several countries. It is worth noting that the market for the TZD type will likely be replaced by other kinds of new drugs.

Taiwanese Patent No. I417088 discloses a chalcone composition for treating diabetes and metabolic diseases. In particular, the chalcone compound bound with 2-halogen in ring A significantly decreases the blood glucose level in in vitro anti-diabetic experiments. In the in vivo animal model, such as mammals, the leading chalcone compound can prevent the progression of diabetes and control the blood glucose level, and there is no significant difference in body weight gain. Throughout the seven-week administration period, no hepatic or renal toxicity was observed in the experiments.

### SUMMARY OF THE INVENTION

The inventor focused on said chalcone compounds and investigated optimization and improvements. It was found that cellular toxicity is generated in cases where there is a functional group with a triple bond in the C6-C3-C6 structure. After optimal modification, the inventor has found a new series of compounds capable of activity against diabetes mellitus without cellular toxicity.

The present invention is related to a composition of an organic compound of a dihydrochalcone skeleton containing C6-C3-C6. A composition of dihydrochalcone for treating diabetes mellitus and metabolic syndromes and a preparation method thereof are disclosed.

In accordance with one aspect of the present invention, a composition of an organic compound of a dihydrochalcone skeleton containing C6-C3-C6 is disclosed. A composition of the dihydrochalcone for treating diabetes mellitus and metabolic syndromes and a preparation method thereof are disclosed.

In accordance with another aspect of the present invention, a compound for treating a metabolic syndrome is disclosed. The compound includes a structure of a 6Cs unit-3Cs unit-6Cs unit (C6-C3-C6) as shown in formula I,

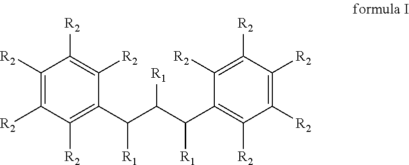

wherein:

when R₁ and R₂ are both hydrogens, C3 has only single bonds;

when R₁ is an oxygen and R₂ is one selected from a group consisting of an alkoxy, a benzyloxy and a halogen, C3 has only single bonds;

when R₁ is one selected from a group consisting of a hydrogen, a deuterium and an oxygen and R₂ is one selected from a group consisting of a hydrogen, an alkoxy, a benzyloxy and a halogen, C3 has one of a single bond and a double bond.

In accordance with another aspect of the present invention, a pharmaceutical composition for treating a metabolic syndrome is disclosed. The pharmaceutical composition includes a compound having a structure of a 6Cs unit-3Cs unit-6Cs unit (C6-C3-C6) as shown in formula II:

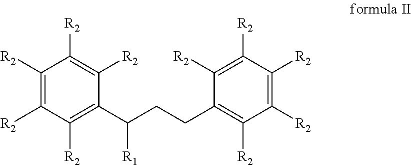

wherein R₁ is one of a deuterium and an oxygen, R₂ is a halogen, and C3 has at least one bond selected from a group consisting of a single bond, a double bond, a triple bond and a combination thereof.

The above objectives and advantages of the present invention will become more readily apparent to those ordinarily skilled in the art after reviewing the following detailed descriptions and accompanying drawings, in which:

### BRIEF DESCRIPTION OF THE DRAWINGS

This patent application contains at least one drawing executed in color. Copies of this patent or patent application publication with color drawings will be disclosed by the Office upon request with payment of the necessary fee.

FIGS. 1-5 illustrate all of the varieties of C6-C3-C6 according to the embodiments of the present invention;

FIGS. 6A-6C illustrate the results of the activity of glucose consumption rate of fat cells (adipocytes);

FIG. 7 illustrates the results of the activity of glucose consumption rate of muscle cells;

FIG. 8 illustrates fat cells and the accumulation of oil droplets under a microscope;

FIG. 9 illustrates the accumulation of oil droplets in fat cells; and

FIGS. 10-12 illustrate that C6-C3-C6 activates the AMPK pathway.

FIGS. 13-20 illustrate the microscopy photos of undifferentialized fat cells under different compounds with the same concentration for 24 hours respectively.

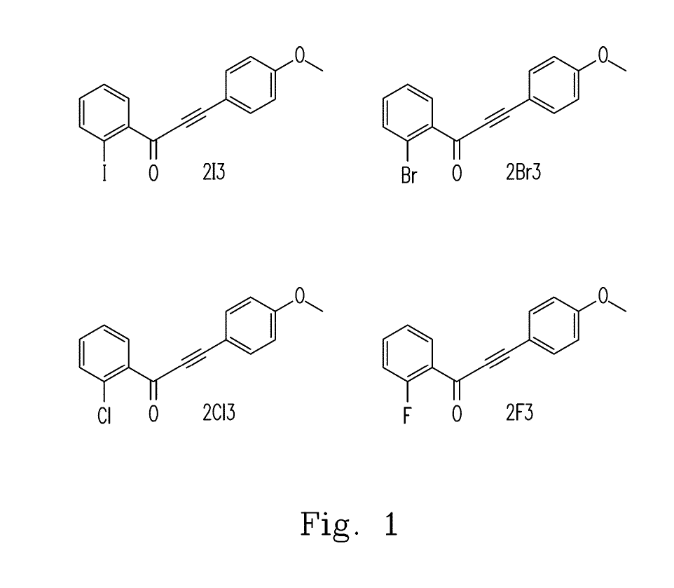

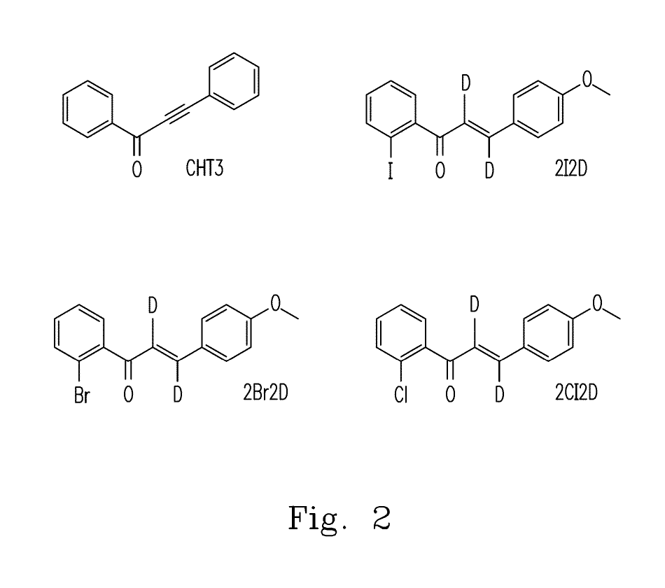

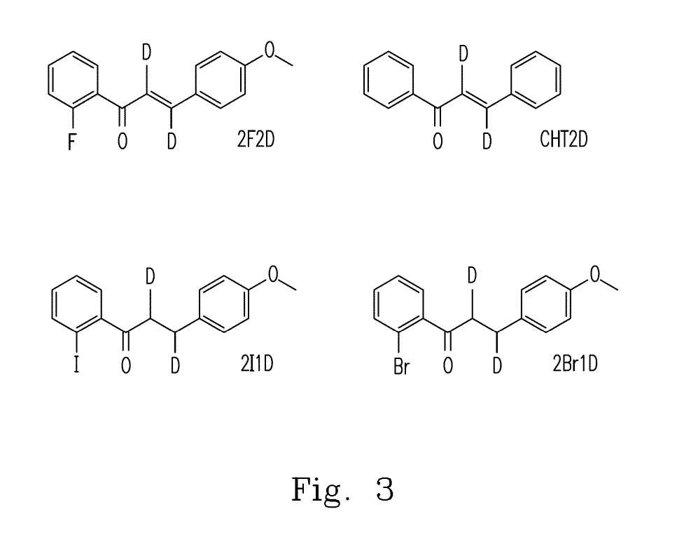

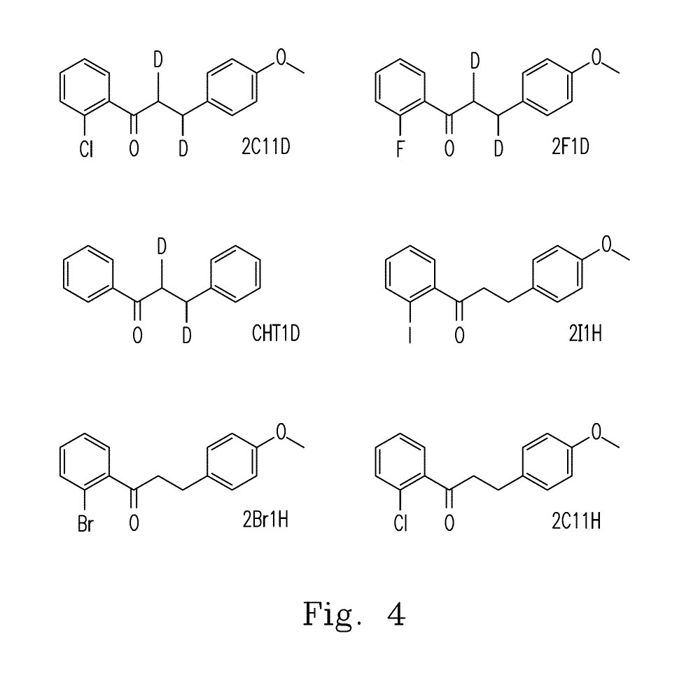

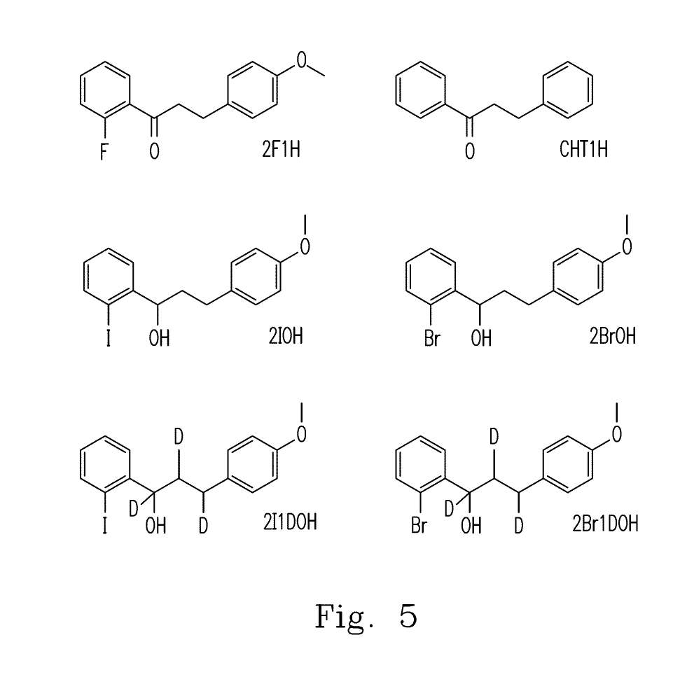

### DETAILED DESCRIPTION OF THE PREFERRED EMBODIMENTS

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

The C6-C3-C6 compound in natural products can be isolated from a plant, such as Chrysanthemum, Lauraceae and Lilium. And the se extracts are classified as flavones. The present application was inspired by the natural compounds, which led to the breakthrough. A halogen element was added to the structure, and this element does not exist in plant extracts. Thus a series of a chalcone type compounds with double bond can be created. It was initially found that the compound containing the halogen promotes the glucose consumption rate in fat cells and that in muscle cells. It was further found that preventing and even improving glucose intolerance due to obesity are possible according to the results of animal experiments.

On the basis of the chalcone, the inventor maintained the C6-C3-C6 skeleton as well as the halogen element in the compound, and then reconstructed a bridge of C3 between two benzene rings. For example, the C3 was partially synthesized to a triple bond compound, or reduced to a single bond compound, and a test of activity was performed.

In addition, pharmaceutical chemists are always interested in stable isotope compounds. They play a very important role in the drugs in bioavailability research. They can not only be used to track the subject, but also to change the metabolic rate of drugs in the human body. However, with old technologies, this research was too expensive to lead to further studies.

Because the present application uses advanced fluid chemistry technologies, a deuterium element can be connected to a specific position of the compound. While greatly reducing the cost, it is possible to conduct further drug research. The inventor further replaced the hydrogen atom linked on the C3 with stable isotopic deuterium using fluid chemistry technologies, and it became a completely new compound.

Combining the above concepts, the inventor further modified the C3 structure, and created more non-natural artificial C6-C3-C6 compounds containing the halogen or deuterium. The inventor designed various methods of synthesizing C6-C3-C6 from organic compounds and modifying structures, and applied fluid chemistry technologies to synthesize new compounds containing the deuterium element.

The following is the method for synthesizing C6-C3-C6 ((I)-(IX)), in which all kinds of C6-C3-C6 according to the embodiments of the present invention are illustrated, including 2I3, 2Br3, 2Cl3, 2F3, CHT3, 2I2D, 2Br2D, 2Cl2D, 2F2D, CHT2D, 2I1D, 2Br1D, 2Cl1D, 2F1D, CHT1D, 2I1H, 2Br1H, 2Cl1H, 2F1H, CHT1H, 2IOH, 2BrOH, 2I1DOH, 2Br1DOH, etc.

(I) The Sonogashira coupling synthesis method was used to manufacture various C6-C3-C6 compounds containing the functional group of triple bonds, in which the C3 structure between the two benzene rings contains a ketone and a triple bond of carbon-carbon (Alkyne), as the following formula shows.

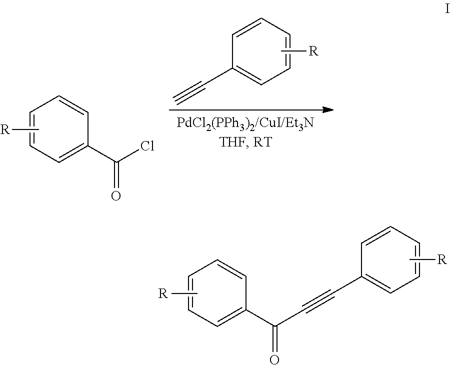

(II) General synthesis method for chalcones with double bonds: the Aldol condensation synthesis method was used to combine Benzaldehyde compounds and Acetophenone compounds, as the following formula shows.

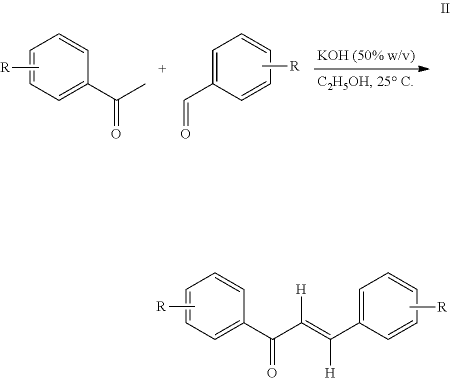

(III) The Deuterium (D₂) was used to reduce the C3 in the structure of the compound containing triple bond in method (I) to obtain a variety of D elements of C6-C3-C6 compounds containing a deuterium (D) element. The first step is to set an initiator in the environment of D₂, and add a metal catalyst to accelerate a deuteride reduction, as the following formula shows.

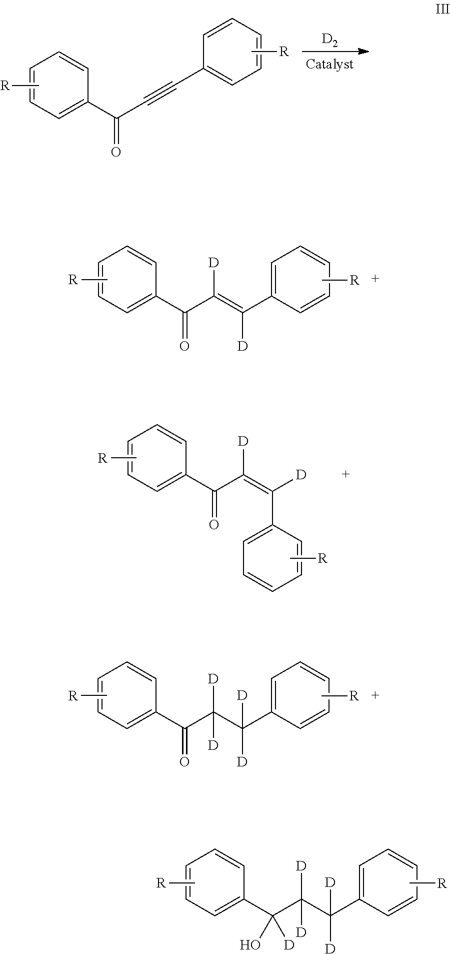

(IV) The Hydrogen gas (H₂) was used to reduce the C3 in the structure of the compound containing triple bonds in method (I) to obtain a variety of C6-C3-C6 compounds containing hydrogen elements. The first step is to set an initiator in the environment of H₂, and add a metal catalyst to accelerate a hydrogenation reduction, as the following formula shows.

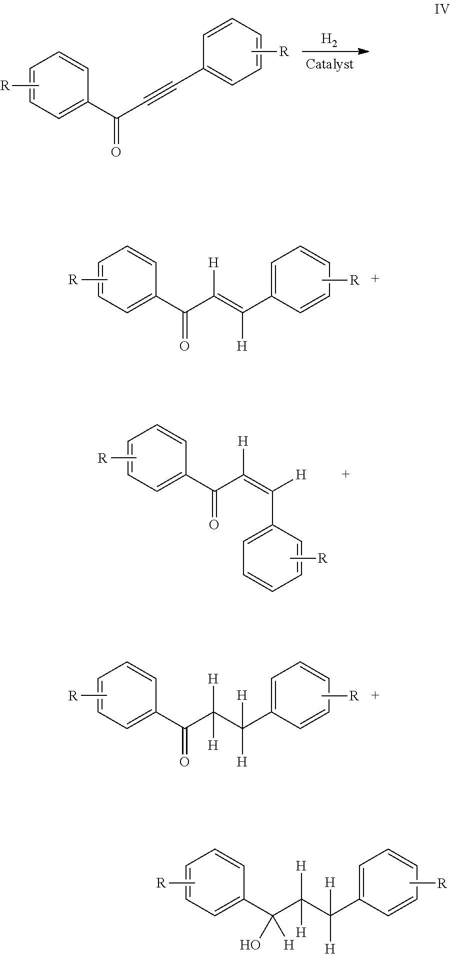

(V) This method is to put chalcone compounds in the environment of D₂, and add a metal catalyst to accelerate a deuteride reduction, and then a compound containing the deuterium different from that of method (III) is obtained, as the following formula shows.

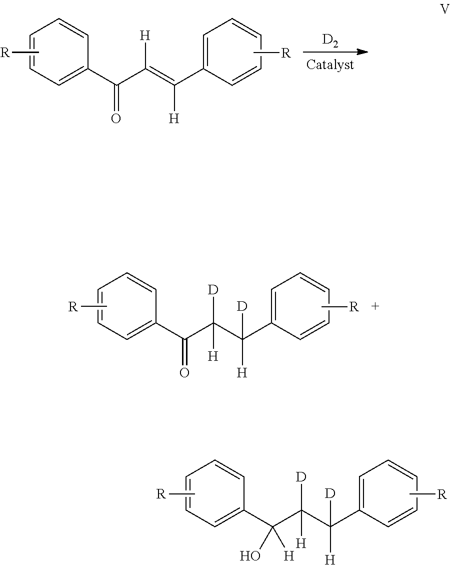

(VI) This method is related to a preparation for trans-β-benzylstyrene derivatives and cis-β-benzylstyrene derivatives, as the following formula shows.

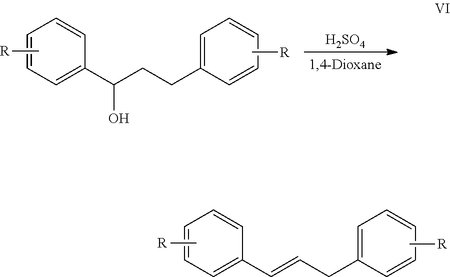

(VII) Preparation methods for trans-β-benzylstyrene derivatives and cis-β-benzylstyrene derivatives containing deuterium are disclosed, as the following formula shows.

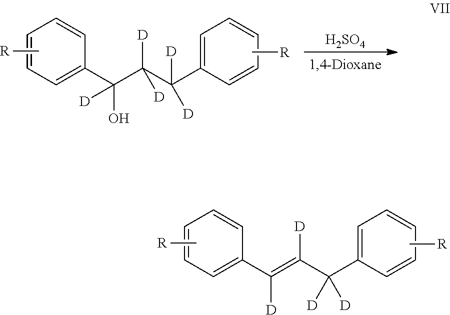

(VIII) Hydrogenation reducing products obtained from Method (VI) to generate 1,3-di-phenylpropane derivatives are disclosed, as the following formula shows.

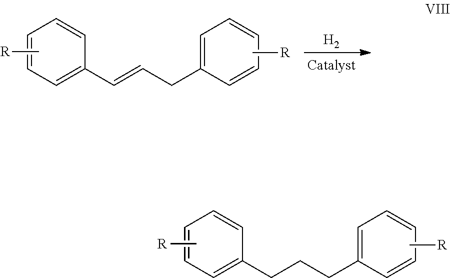

(IX) Deuteride reducing products obtained from method (VII) to generate 1,3-di-phenylpropane derivatives containing the deuterium element are disclosed, as the following formula shows.

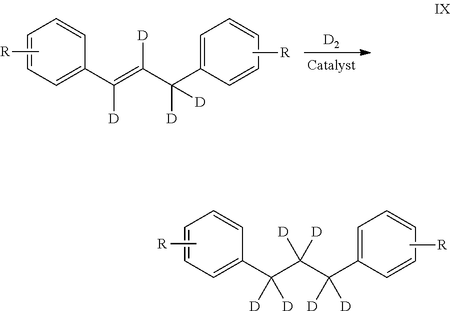

An embodiment of a method for synthesizing a deuterium containing compound:

A Preparation for 2,3-dideutero-1-(2-iodophenyl)-3-(4-methoxyphenyl)propan-1-one:

The preparation method applies an H-Cube® system, which could electrolyses water into oxygen and hydrogen, and propagate the hydrogen gas into the pipeline. An initiator and the hydrogen gas are fully mixed, and the mixed is treated with a metal catalyst to generate a hydrogenation reduction.

(E)-1-(2-iodophenyl)-3-(4-methoxyphenyl)prop-2-en-1-one (2I₂H) is applied as the initiator in this example, and it is dissolved in ethyl acetate (1 mg/ml). 5% Pt/Al₂O₃ and 5% Pd/BaSO₄ are used as the metal catalyst. Then an electrolysis of D₂O to produce deuterium (D₂) is performed. The pressure and temperature are manipulated to perform a deuteride reduction.

It was found that 5% Pd/BaSO₄ cannot start the reduction of the initiator, and thus 5% Pt/Al₂O₃ is applied instead. After optimizing the reaction conditions to 100° C., pressure of 100 bar and flow velocity of 1 ml/min, a product “2,3-dideutero-1-(2-iodophenyl)-3-(4-methoxyphenyl)propan-1-one (2I1D)” was generated at a conversion rate of 100%.

Physical Data:

2,3-dideutero-1-(2-iodophenyl)-3-(4-methoxyphenyl)propan-1-one (2I1D):

The light-yellow oil compound: ¹H NMR (400 MHz, CHLOROFORM-d), δ=2.96-2.98 (m, 1 H), 3.15-3.17 (m, 1 H), 3.77 (s, 3 H), 6.82 (d, J=8.81 Hz, 2 H), 7.09 (t, J=7.62 Hz, 1 H), 7.14 (d, J=8.56 Hz, 2 H), 7.30 (dd, J=7.68, 1.64 Hz, 1 H), 7.36 (t, J=7.60 Hz, 1 H), 7.89 ppm (d, J=8.06 Hz, 1 H); ¹³C NMR (101 MHz, CHLOROFORM-d), δ=28.81, 43.86, 55.23, 90.91, 113.88 (2 C), 127.74, 127.99, 129.35 (2 C), 131.53, 132.68, 140.49, 144.52, 157.99, 204.07 ppm; MS (EI): m/z (%): 367.91, 241.13, 122.11, 109.14.

Physical Data for CHT1D:

2,3-dideutero-1,3-diphenylpropan-1-one:

The colorless oil compound: ¹H NMR (400 MHz, CHLOROFORM-d, 25° C., TMS): δ=3.07 (br. s., 1 H), 3.21-3.35 (m, 1 H), 7.16-7.41 (m, 5 H, and solvent peak), 7.44-7.48 (m, 2 H), 7.54-7.58 (m, 1 H), 7.97 ppm (d, J=7.55 Hz, 2 H); ¹³C NMR (101 MHz, CHLOROFORM-d, 25° C., TMS), δ=29.51, 39.69, 126.04, 127.96 (2 C), 128.32 (2 C), 128.44 (2 C), 128.50 (2 C), 132.94, 136.81, 141.15, 199.16 ppm. MS (EI): m/z (%): 212.06, 105.05, 77.1, 51.09.

Physical Data for 2F1D:

2,3-dideutero-1-(2-florophenyl)-3-(4-methoxyphenyl)propan-1-one (2dA):

The colorless oil compound: ¹H NMR (400 MHz, CHLOROFORM-d), δ=2.98-2.99 (m, 1 H), 3.27-3.29(m, 1 H), 3.80 (s, 3 H), 6.85 (d, J=8.56 Hz, 2 H), 7.11-7.27 (m, 4 H), 7.51-7.52 (m, 1 H), 7.84-7.88 ppm (m, 1 H); ¹³C NMR (101 MHz, CHLOROFORM-d) δ32 28.67, 45.03, 55.18, 113.81 (2 C), 116.72, 124.41, 129.32 (2 C), 130.61, 133.06, 134.00, 134.44, 157.81, 163.1, 197.79 ppm; MS (EI): m/z (%): 260.07, 122.10, 109.13.

Physical Data for 2Cl1D:

2,3-dideutero-1-(2-chlorophenyl)-3-(4-methoxyphenyl)propan-1-one:

The colorless oil compound: ¹H NMR (400 MHz, CHLOROFORM-d) δ=2.97-2.98 (m, 1 H), 3.20-3.22 (m, 1 H), 3.77 (s, 3 H), 6.83 (d, J=8.56 Hz, 2 H), 7.14 (d, J=8.56 Hz, 2 H), 7.27-7.41 ppm (m, 4 H); ¹³C NMR (101 MHz, CHLOROFORM-d), δ=28.82, 44.26, 55.11, 113.78 (2 C), 126.81, 128.81, 129.22 (2 C), 130.37, 130.73, 131.58, 132.61, 139.25, 157.88, 202.60 ppm; MS (EI): m/z (%): 276.09, 241.14, 139.01, 122.11, 109.14.

Physical Data for 2Br1D:

2,3-dideutero-1-(2-bromophenyl)-3-(4-methoxyphenyl)propan-1-one (4dA):

The light-yellow oil compound: ¹H NMR (400 MHz, CHLOROFORM-d), δ=3.02-3.04 (m, 1 H), 3.23-3.25 (m, 1 H), 3.82 (s, 3 H), 6.87 (d, J=8.56 Hz, 2 H), 7.18 (d, J=8.31 Hz, 2 H), 7.30-7.37 (m, 3 H), 7.63 ppm (d, J=7.55 Hz, 1 H); ¹³C NMR (101 MHz, CHLOROFORM-d), δ=28.74, 44.36, 55.11, 113.77 (2 C), 118.51, 127.30, 128.33, 129.23 (2 C), 131.43, 132.54, 133.49, 141.51, 157.87, 203.44 ppm; MS (EI): m/z (%): 321.96, 319.98, 241.14, 240.12, 184.99, 183.00, 122.12, 108.12.

Physical Data for 2I1D:

2,3-dideutero-1-(2-iodophenyl)-3-(4-methoxyphenyl)propan-1-one (5dA):

The light-yellow oil compound: ¹H NMR (400 MHz, CHLOROFORM-d), δ=2.96-2.98 (m, 1 H), 3.15-3.17 (m, 1 H), 3.77 (s, 3 H), 6.82 (d, J=8.81 Hz, 2 H), 7.09 (t, J=7.62 Hz, 1 H), 7.14 (d, J=8.56 Hz, 2 H), 7.30 (dd, J=7.68, 1.64 Hz, 1 H), 7.36 (t, J=7.60 Hz, 1 H), 7.89 ppm (d, J=8.06 Hz, 1 H); ¹³C NMR (101 MHz, CHLOROFORM-d), δ=28.81, 43.86, 55.23, 90.91, 113.88 (2 C), 127.74, 127.99, 129.35 (2 C), 131.53, 132.68, 140.49, 144.52, 157.99, 204.07 ppm; MS (EI): m/z (%): 367.91, 241.13, 122.11, 109.14.

Statistical Evaluation of Data:

The results were expressed as mean±SE. Statistical differences were determined by independent and paired students' t-tests in unpaired and paired samples. When a control group was compared with more than one treated group, one-way ANOVA or two-way repeated measure ANOVA was used. When the ANOVA showed a statistical difference, the Dunnett's or Student-Newman-Keuls test was applied. A P value less than 0.05 was considered significant in all experiments. Analysis of the data and plotting of the figures were done with the aid of SigmaPlot software (Version 8.0, Chicago, Ill., U.S.A.) and SigmaStat (Version 2.03, Chicago, Ill., U.S.A.) run on an IBM compatible computer.

The toxicity and biological activity with various cell modes was used to find more potential compounds. Structure-activity relationship (SAR) studies show that certain compounds can not only regulate the glucose consumption rate of fat cells and muscle cells, but also its pathway of metabolism.

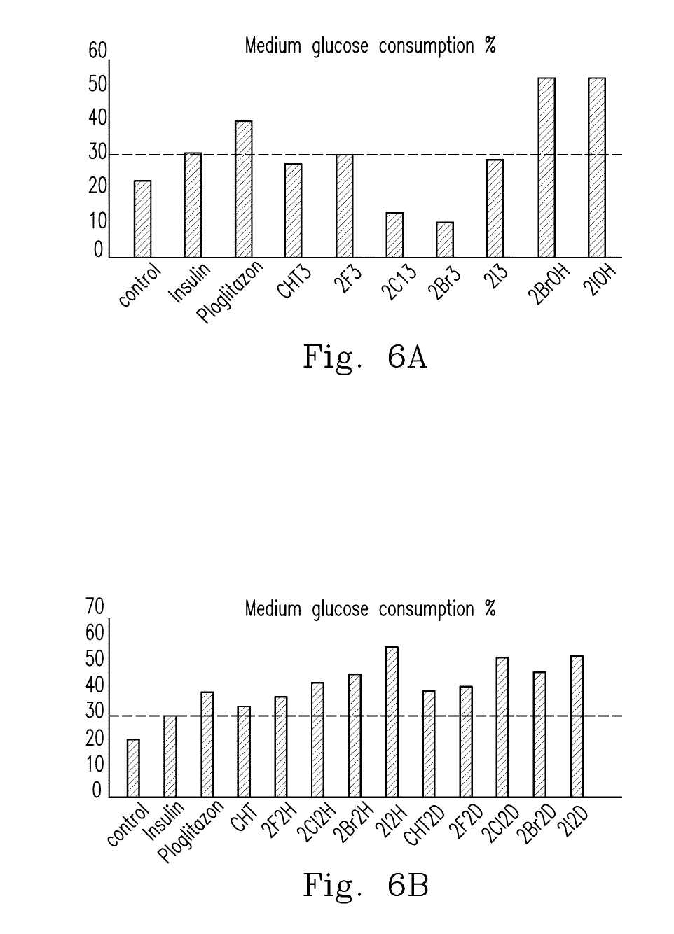

Please refer to FIGS. 6A-6C. FIGS. 6A-6C show the results of the activity of the glucose consumption rate of fat cells in the culture medium. As shown in FIGS. 6A-6C, various compounds containing halogens, such as chlorine, bromine and iodine, can increase the glucose consumption rate of fat cells. Many of the deuterium-containing compounds are new ones in this example. Many known compounds demonstrated this activity for the first time. Symbol ‘Con’ denotes the Control Group, Symbol ‘5’ denotes a 2Br2H compound, Symbol ‘6’ denotes a 2I2H compound, Symbol ‘Met’ denotes a metformin, Symbol ‘AI’ denotes an AMPK inhibitor, Symbol ‘L’ denotes a low-dose usage of 15 ug/mL, and Symbol ‘H’ denotes a high-dose usage of 30 ug/mL

The activity of the glucose consumption rate of fat cells is taken from the following: A testing drug, 2,3-dideutero-1-(2-iodophenyl)-3-(4-methoxyphenyl)propan-1-one with the concentration 30 ug/mL, was cultured with mature fat cells for 24 hours. It was then tested to determine the change of the glucose concentration in the culture medium.

24 hours after the replacement of a culture media, the glucose consumption rate of the control group was about 20%. After insulin was added, the glucose consumption rate of the cells in the control group upgrades to 30%. With the commercial drug, Pioglitazone of 30 ug/mL, the glucose consumption rate of the cells in the corresponding group increased to 40%.

Please continue to refer to FIG. 6A, in which the compound 1-(2-iodophenyl)-3-(4-methoxyphenyl)prop-2-yn-1-one (2I3) containing a triple bond structure produces significant cellular toxicity. The glucose consumption rate of the cells in the group with an initiator of a double bond compound, 2I2H, may exceed 50%. The glucose consumption rate of the cells in the group with the addition of the product of a single bond compound, 2I1D, also exceeds 50%. When the concentration of the drug is 30 ug/mL, there is no visible cellular toxicity for compounds 2I2H and 2I1D, and thus the reduced structures could reduce the problem of cellular toxicity, and generate or maintain the activity of the glucose consumption rate.

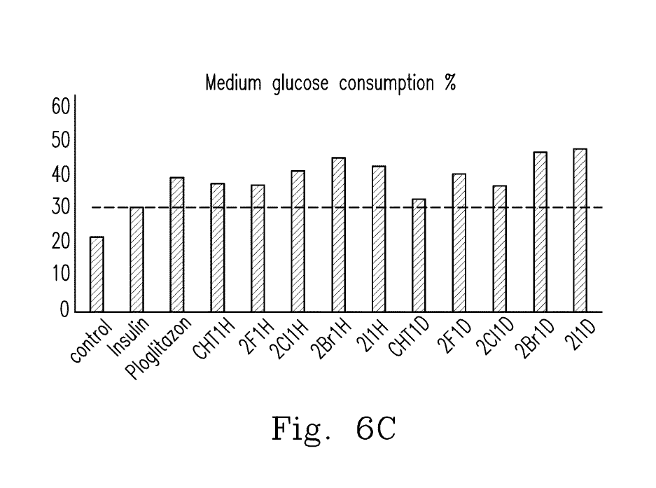

Please refer to FIG. 7. FIG. 7 shows the results of the activity of the glucose consumption rate in muscle cells. As shown in FIG. 7, various compounds containing halogens, such as chlorine, bromine and iodine, can increase the glucose consumption rate in muscle cells. Many deuterium-containing compounds are new ones in this example. Many known compounds disclosed this activity for the first time. Symbol ‘Con’ denotes a Control Group, Symbol ‘Ins’ denotes an Insulin Group, Symbol ‘Rosi’ denotes a Rosiglitazone Group, Symbol ‘Pio’ denotes a Pioglitazone Group, Symbol ‘1’ denotes a CHT compound, Symbol ‘2’ denotes a 2OH2H compound, Symbol ‘3’ denotes a 2F2H compound, Symbol ‘4’ denotes a 2Cl2H compound, Symbol ‘5’ denotes a 2Br2H compound, and Symbol ‘6’ denotes a 2I2H compound.

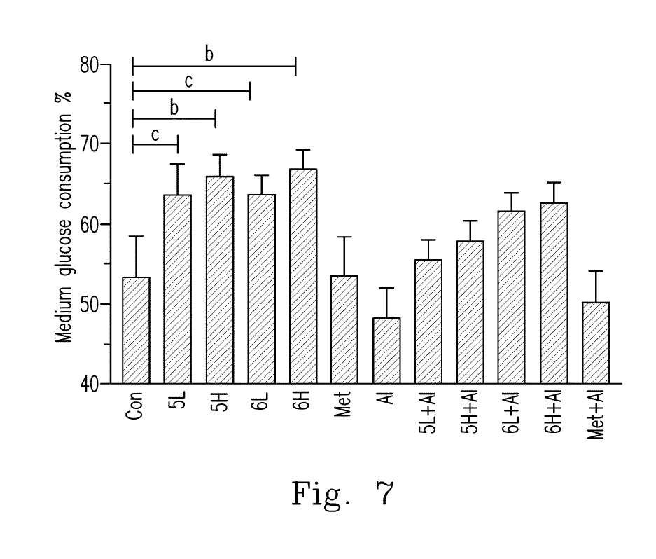

Please refer to FIG. 8, which illustrates fat cells and the accumulation of oil droplets under a microscope. ‘Con’ denotes a Control Group, Symbol ‘Ins’ denotes an Insulin Group, Symbol ‘Rosi’ denotes a Rosiglitazone Group, Symbol ‘Pio’ denotes a Pioglitazone Group, Symbol ‘1’ denotes a CHT compound, Symbol ‘2’ denotes a 2OH2H compound, Symbol ‘3’ denotes a 2F2H compound, Symbol ‘4’ denotes a 2Cl2H compound, Symbol ‘5’ denotes a 2Br2H compound, and Symbol ‘6’ denotes a 2I2H compound. It can be seen that fat cells maintain normal morphologies in each treated group, and thus it is deduced that a low-concentration dosage shows no significant cellular toxicity for all of the compounds disclosed in the present invention.

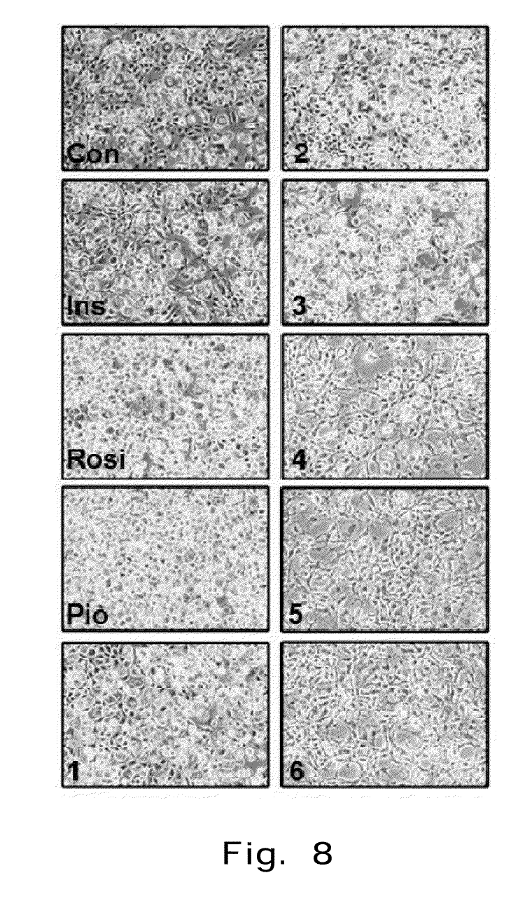

Please refer to FIGS. 8-9. These figures illustrate the accumulation of oil droplets in fat cells in the culture medium. As shown in FIGS. 8-9, various compounds containing halogens, such as chlorine, bromine and iodine, can increase the glucose consumption rate of fat cells without increasing the accumulation of oil droplets in fat cells. Many deuterium-containing compounds are new ones in this example. Many of known compounds demonstrated this activity for the first time. ‘Con’ denotes a Control Group, Symbol ‘Ins’ denotes an Insulin Group, Symbol ‘Rosi’ denotes a Rosiglitazone Group, Symbol ‘Pio’ denotes a Pioglitazone Group, Symbol ‘1’ denotes a CHT compound, Symbol ‘2’ denotes a 2OH2H compound, Symbol ‘3’ denotes a 2F2H compound, Symbol ‘4’ denotes a 2Cl2H compound, Symbol ‘5’ denotes a 2Br2H compound, and Symbol ‘6’ denotes a 2I2H compound.

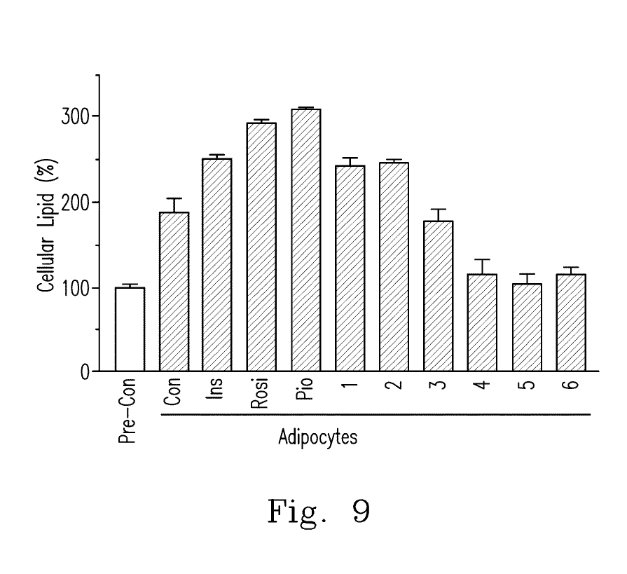

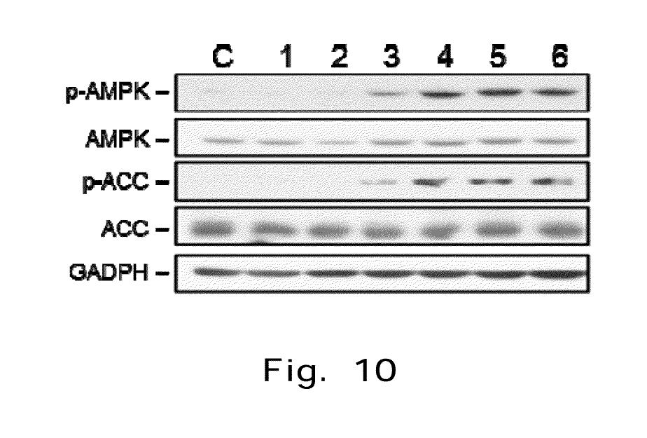

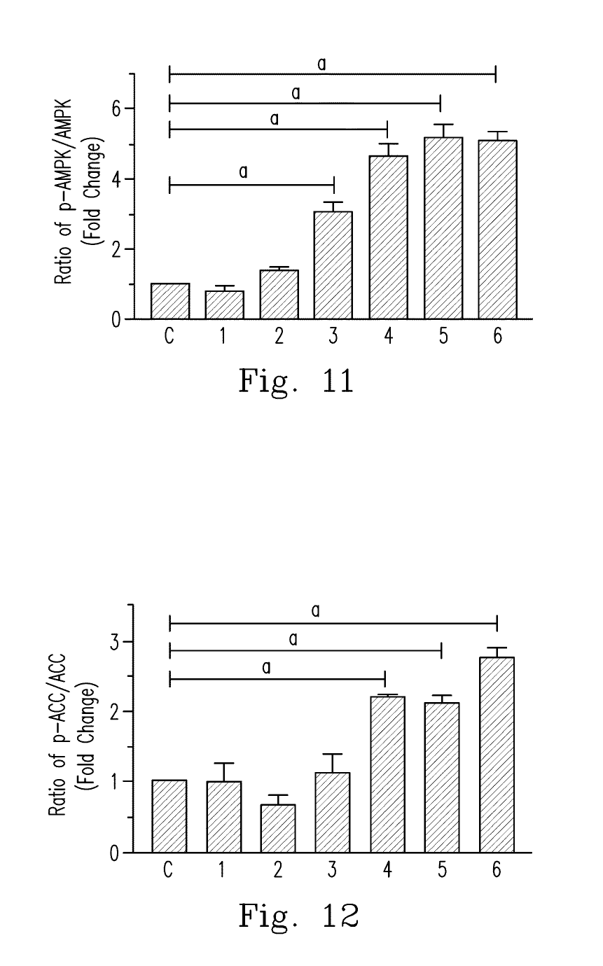

Please refer to FIGS. 10-12. These figures illustrate that C6-C3-C6 activates the AMPK pathway, increases the activity of the glucose consumption rate, and regulates energy use without accumulating oil droplets in the cells. This further affects insulin resistance and improves the effect against metabolic syndromes.

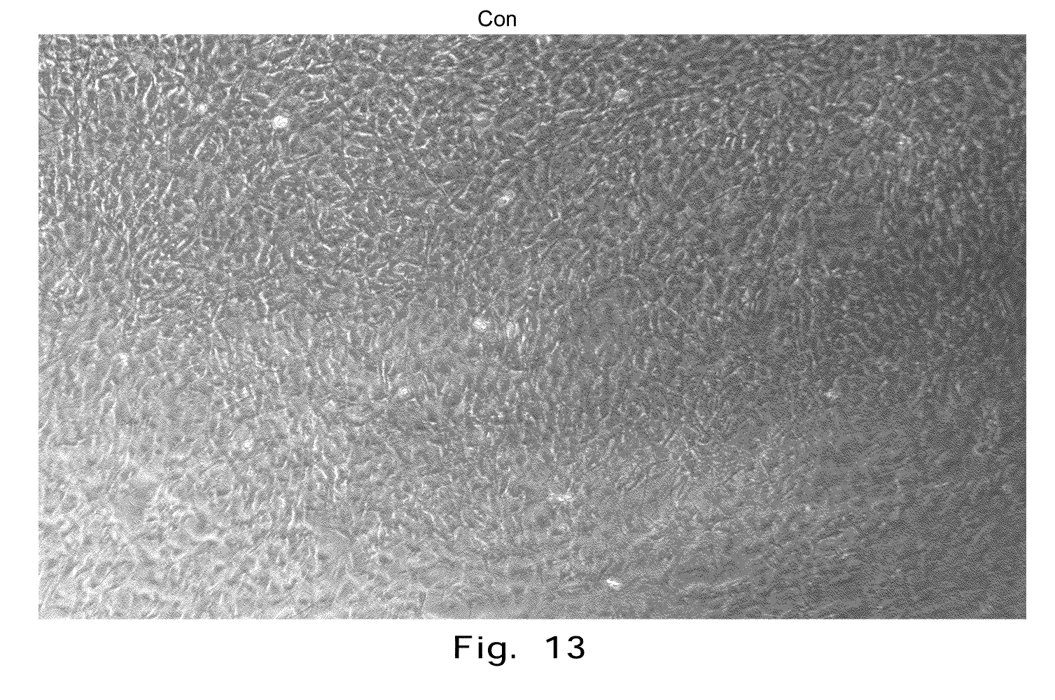

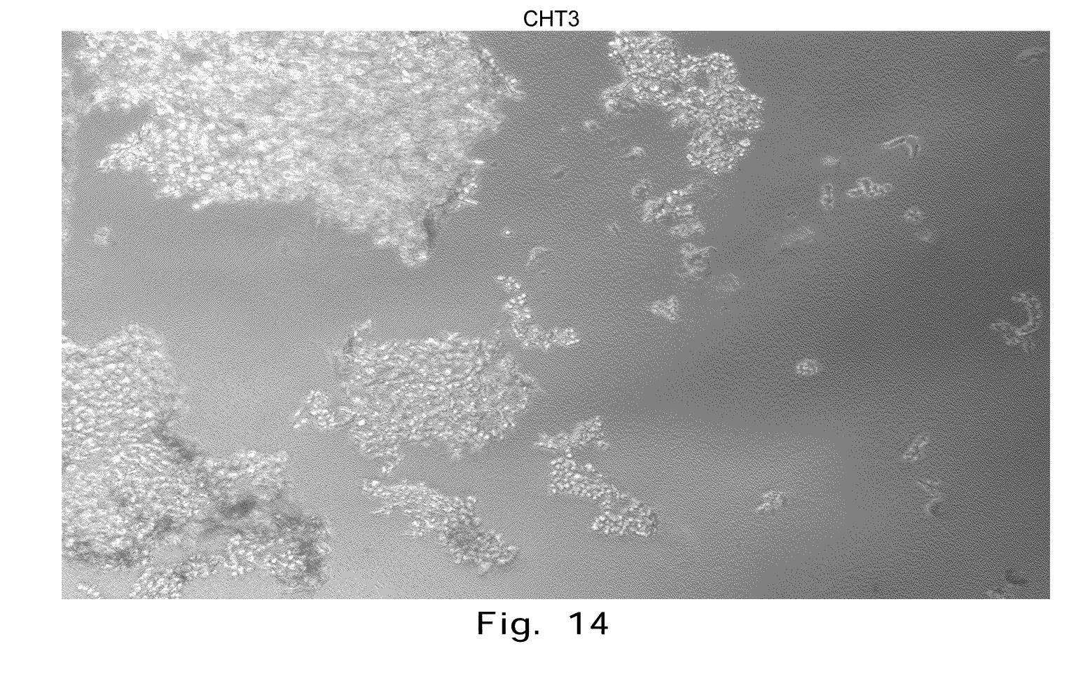

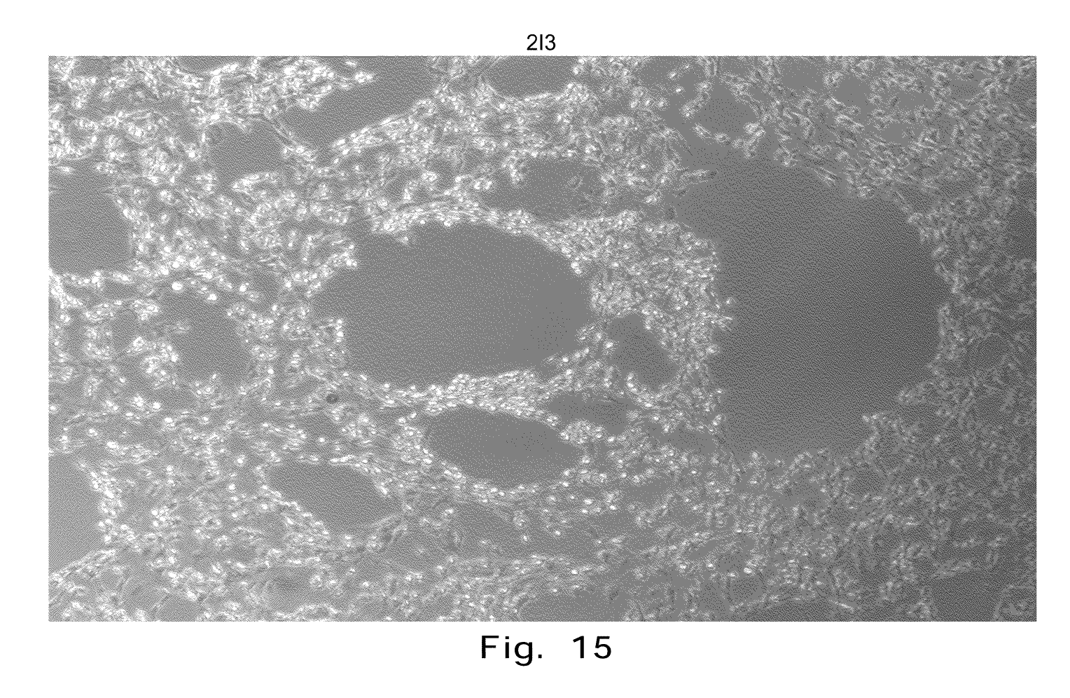

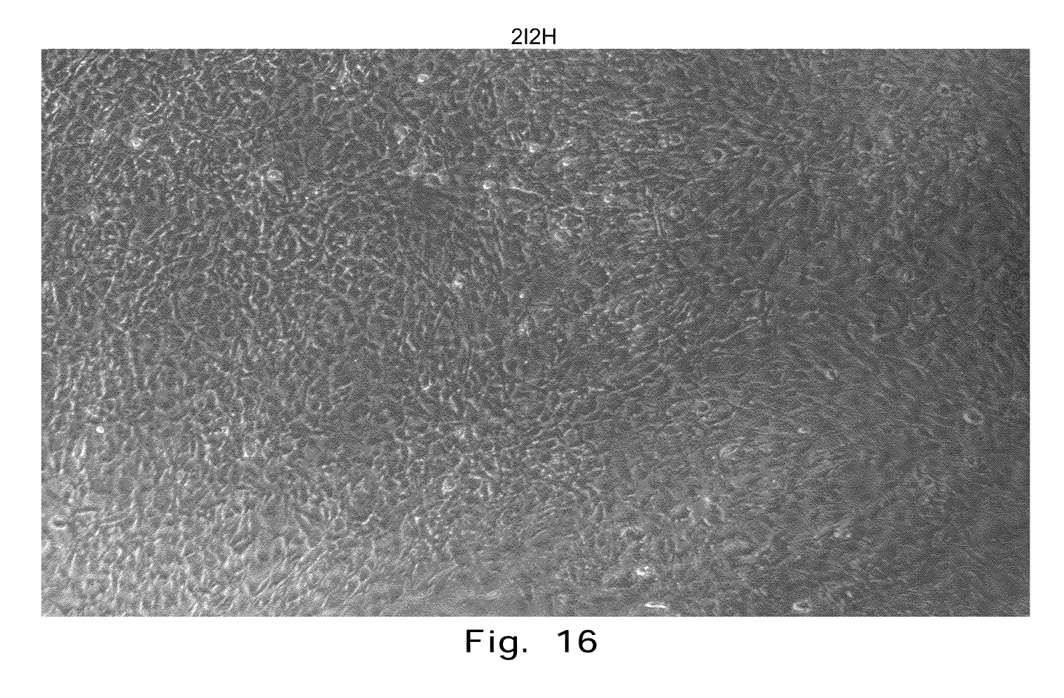

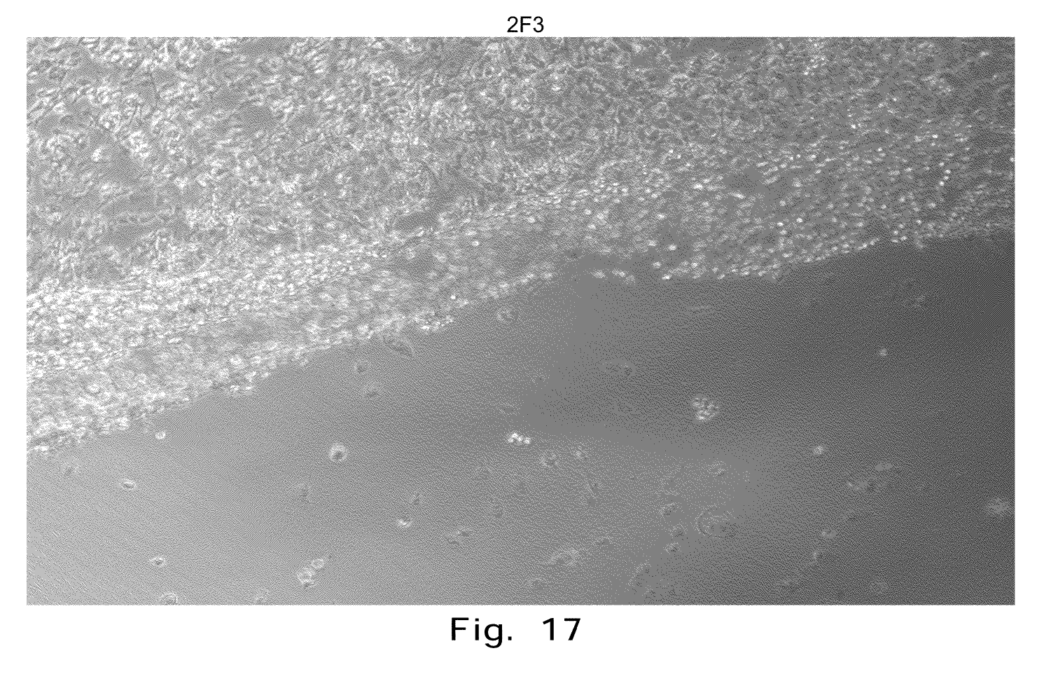

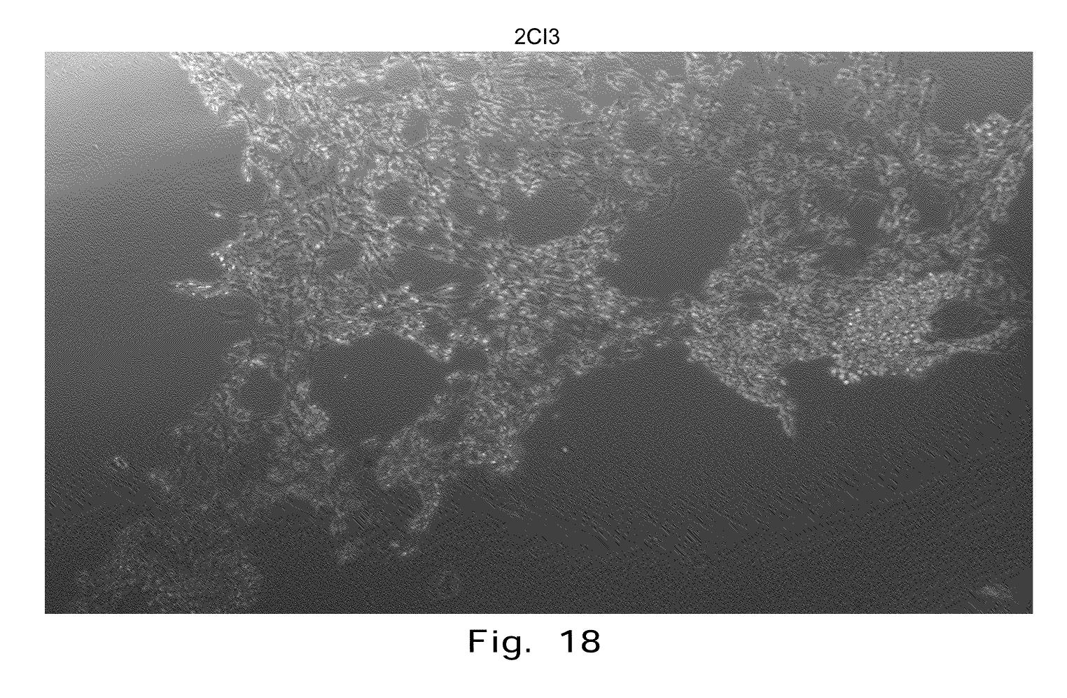

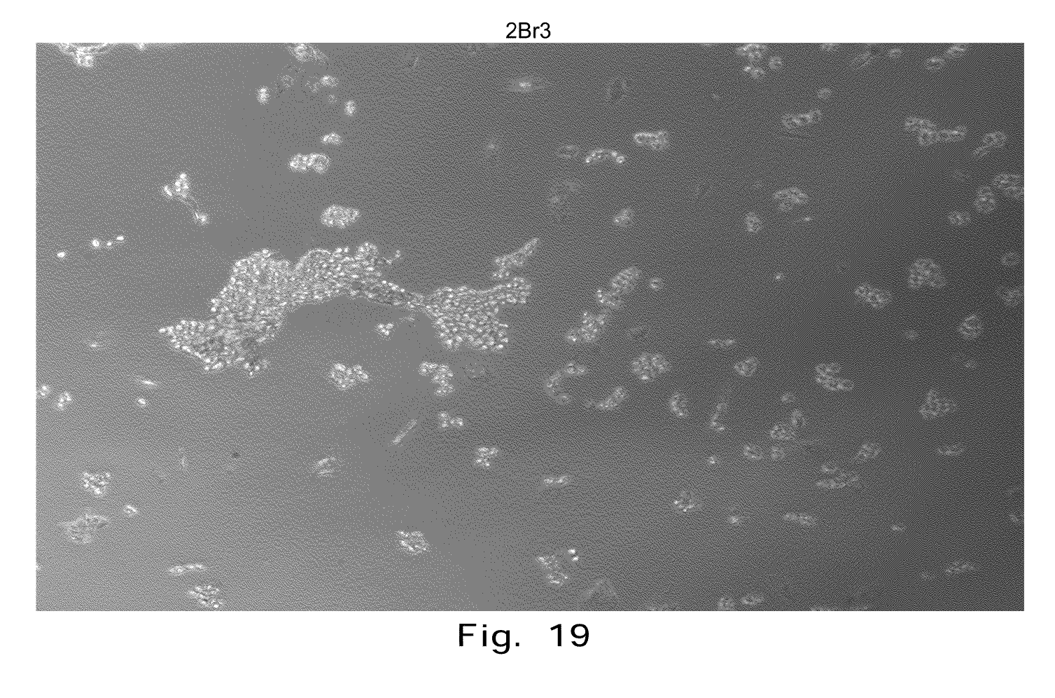

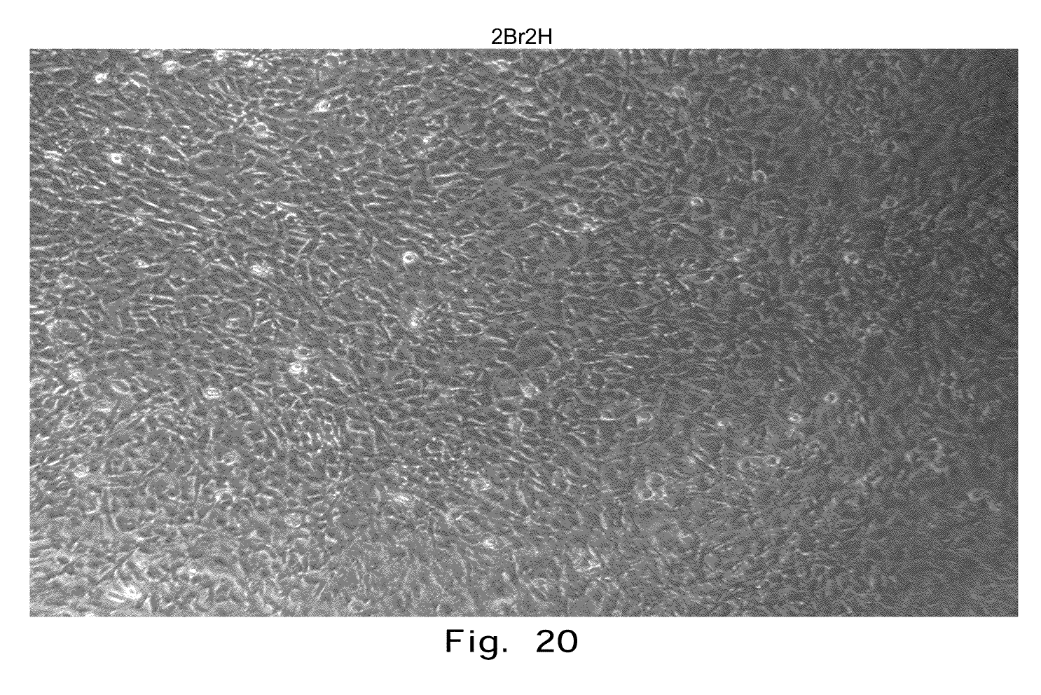

Please refer to FIGS. 13-20, which illustrate the microscopy photos of undifferentialized fat cells under different compounds with the same concentration for 24 hours, control, CHT3, 2I3, 2I2H, 2F3, 2Cl3, 2Br3 and 2Br2H respectively. Compared to complete morphology of the control, there are lots of fragments of the undifferentialized fat cells in the microscopy photos corresponding to treatments with different compounds. Thus it is found that most of the above compounds containing triple bonds show certain cellular toxicity to the undifferentialized fat cells.

In summary, the present application discloses the C6-C3-C6 skeleton of the organic compound, containing a substituent of a halogen or a stable isotope. Therefore, it is completely different from natural compounds, and thus is novel.

A Structure-activity relationship (SAR) study shows that certain compounds in the present invention can not only regulate the glucose consumption rate of fat cells and that of muscle cells and its pathway of metabolism, in which the mechanisms of the activity are not the same as TZD-like drugs, but can also reduce cellular toxicity. Thus the present invention has novelty and progressiveness. Future developments in drug design and the pharmaceutical industry are possible.

After an activity test, it was found that the compound containing the deuterium element shows the same degree of biological activity. Compared with the triple bond compound, the cellular toxicity was greatly reduced. Current-marketed drugs contain no deuterium, and thus the present invention is highly novel, progressive and has medical uses.

It was found that the triple bond compound results in cellular toxicity in fat cells, but the single bond compound maintains the activity of drugs and reduces cellular toxicity. The single bond compound “C6-C3-C6” containing halogen had not been noticed capable of regulating the activity of the glucose consumption rate of cells. Thus the present invention has novelty and progressiveness.

### Embodiments

1. A compound for treating a metabolic syndrome, including a structure of a 6Cs unit-3Cs unit-6Cs unit (C6-C3-C6) as shown in formula I,

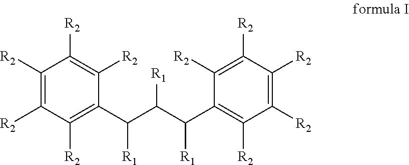

wherein:

when R₁ and R₂ are both hydrogens, C3 has only single bonds;

when R₁ is an oxygen and R₂ is one selected from a group consisting of an alkoxy, a benzyloxy and a halogen, C3 has only single bonds;

when R₁ is one selected from a group consisting of a hydrogen, a deuterium and an oxygen and R₂ is one selected from a group consisting of a hydrogen, an alkoxy, a benzyloxy and a halogen, C3 has one of a single bond and a double bond.

2. The compound as in Embodiment 1, wherein the compound is used to regulate and stabilize a blood glucose level in a cell.

3. The compound as in Embodiment 1, wherein the compound is used to inhibit a glucose intolerance in a cell.

4. The compound as in Embodiment 1, wherein treating the metabolic syndrome includes one of inhibiting and delaying an onset of the metabolic syndrome in a cell.

5. The compound as in Embodiment 1, wherein the metabolic syndrome is related to a diabetes mellitus.

6. A pharmaceutical composition for treating a metabolic syndrome, including a compound having a structure of a 6Cs unit-3Cs unit-6Cs unit (C6-C3-C6) as shown in formula II:

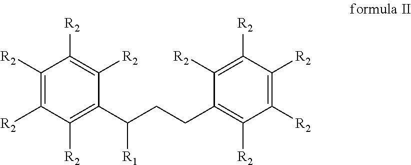

wherein R₁ is one of a deuterium and an oxygen, R₂ is a halogen, and C3 has at least one bond selected from a group consisting of a single bond, a double bond, a triple bond and a combination thereof.

7. The pharmaceutical composition as in Embodiment 6, wherein the pharmaceutical composition is used to regulate and stabilize a blood glucose level in a cell.

8. The pharmaceutical composition as in Embodiment 6, wherein the pharmaceutical composition is used to inhibit a glucose intolerance in a cell.

9. The pharmaceutical composition as in Embodiment 6, wherein treating the metabolic syndrome includes one of inhibiting and delaying an onset of the metabolic syndrome in a cell.

10. The pharmaceutical composition as in Embodiment 6, wherein the metabolic syndrome is related to a diabetes mellitus.

11. The pharmaceutical composition as in Embodiment 6, wherein the C6-C3-C6 is a chalcone.

12. The pharmaceutical composition as in Embodiment 6, wherein the deuterium is prepared by a hydrogenation reduction.

13. The pharmaceutical composition as in Embodiment 6, wherein the triple bond is prepared by an Aldol condensation synthesis.

14. The pharmaceutical composition as in Embodiment 6, wherein the double bond is prepared by a Sonogashira coupling synthesis.

15. The pharmaceutical composition as in Embodiment 6, further including an ingredient selected from a group consisting of a pharmaceutically acceptable salt, solvate and the combination thereof.

16. A method for treating a metabolic syndrome, including a step of:

administrating a pharmaceutical composition having one of the compounds according to claim 1 or claim 6 to a mammal

17. The method as in Embodiment 16, wherein the method is used to regulate and stabilize a blood glucose level in a cell.

18. The method as in Embodiment 16, wherein the method is used to inhibit a glucose intolerance in a cell.

19. The method as in Embodiment 16, wherein treating the metabolic syndrome includes one of inhibiting and delaying an onset of the metabolic syndrome in a cell.

20. The method as in Embodiment 16, wherein the metabolic syndrome is related to a diabetes mellitus.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A compound for treating a metabolic syndrome, comprising a structure of a 6Cs unit-3Cs unit-6Cs unit (C6-C3-C6) as shown in formula I, 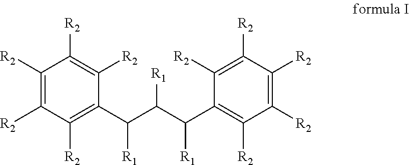
wherein:
when R₁ and R₂ are both hydrogens, C3 has only single bonds;
when R₁ is an oxygen and R₂ is one selected from a group consisting of an alkoxy, a benzyloxy and a halogen, C3 has only single bonds;
when R₁ is one selected from a group consisting of a hydrogen, a deuterium and an oxygen and R₂ is one selected from a group consisting of a hydrogen, an alkoxy, a benzyloxy and a halogen, C3 has one of a single bond and a double bond.

2. The compound as claimed in claim 1, wherein the compound is used to regulate and stabilize a blood glucose level in a cell.

3. The compound as claimed in claim 1, wherein the compound is used to inhibit a glucose intolerance in a cell.

4. The compound as claimed in claim 1, wherein treating the metabolic syndrome includes one of inhibiting and delaying an onset of the metabolic syndrome in a cell.

5. The compound as claimed in claim 1, wherein the metabolic syndrome is related to a diabetes mellitus.

6. A pharmaceutical composition for treating a metabolic syndrome, comprising a compound having a structure of a 6Cs unit-3Cs unit-6Cs unit (C6-C3-C6) as shown in formula II: 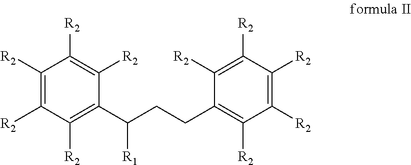
wherein R₁ is one of a deuterium and an oxygen, R₂ is a halogen, and C3 has at least one bond selected from a group consisting of a single bond, a double bond, a triple bond and a combination thereof.

7. The pharmaceutical composition as claimed in claim 6, wherein the pharmaceutical composition is used to regulate and stabilize a blood glucose level in a cell.

8. The pharmaceutical composition as claimed in claim 6, wherein the pharmaceutical composition is used to inhibit a glucose intolerance in a cell.

9. The pharmaceutical composition as claimed in claim 6, wherein treating the metabolic syndrome includes one of inhibiting and delaying an onset of the metabolic syndrome in a cell.

10. The pharmaceutical composition as claimed in claim 6, wherein the metabolic syndrome is related to a diabetes mellitus.

11. The pharmaceutical composition as claimed in claim 6, wherein the C6-C3-C6 is a chalcone.

12. The pharmaceutical composition as claimed in claim 6, wherein the deuterium is prepared by a hydrogenation reduction.

13. The pharmaceutical composition as claimed in claim 6, wherein the triple bond is prepared by an Aldol condensation synthesis.

14. The pharmaceutical composition as claimed in claim 6, wherein the double bond is prepared by a Sonogashira coupling synthesis.

15. The pharmaceutical composition as claimed in claim 6, further comprising an ingredient selected from a group consisting of a pharmaceutically acceptable salt, solvate and the combination thereof.

16. A method for treating a metabolic syndrome, comprising a step of: administrating a pharmaceutical composition having one of the compounds according to claim 1 or claim 6 to a mammal

17. The method as claimed in claim 16, wherein the method is used to regulate and stabilize a blood glucose level in a cell.

18. The method as claimed in claim 16, wherein the method is used to inhibit a glucose intolerance in a cell.

19. The method as claimed in claim 16, wherein treating the metabolic syndrome includes one of inhibiting and delaying an onset of the metabolic syndrome in a cell.

20. The method as claimed in claim 16, wherein the metabolic syndrome is related to a diabetes mellitus.
